(19) United States
(12) Patent Application Publication
(10) Pub. No.: US 2026/0232692 A1
(43) Pub. Date: Aug. 13, 2026

(54) Methods Of Treating Cancer Using BCL-2 Inhibitors With Iron And Heme Supplementation Therapies

(71) Applicant: Emory University, Atlanta, GA (US)

(72) Inventors: Malathy Shanmugam, Atlanta, GA (US); Remya Nair, Atlanta, GA (US)

(21) Appl. No.: 19/469,899

(22) PCT Filed: Mar. 28, 2024
(86) PCT No.: PCT/US2024/022030

§ 371 Date: Sep. 26, 2025

Related U.S. Application Data
(60) Provisional application No. 63/455,110, filed on Mar. 28, 2023.
(60) Provisional application No. 63/581,134, filed on Sep. 7, 2023.

(51) Int. Cl. A61K 31/555; A61K 31/517; A61K 31/635; A61K 33/26; A61K 45/06; A61P 35/00

(52) U.S. Cl. CPC A61K 31/555; A61K 31/517; A61K 31/635; A61K 33/26; A61K 45/06; A61P 35/00

(57) ABSTRACT

Disclosed herein are methods of managing cancer treatments using an anticancer agent and iron, heme, iron complexes, and/or iron salts. In certain embodiments, this disclosure relates to methods of treating cancer comprising administering to a patient in need thereof an effective amount of a BCL-2 inhibitor in combination with iron, heme, iron complex, and/or iron salt. In certain embodiments, the cancer is a hematological malignancy. In certain embodiments, the iron complex is a heme iron complex.

### CROSS-REFERENCE TO RELATED APPLICATIONS

This application claims the benefit of U.S. Provisional Application No. 63/455,110 filed Mar. 28, 2023 and U.S. Provisional Application No. 63/581,134 filed Sep. 7, 2023. The entirety of each of these applications is hereby incorporated by reference for all purposes.

### BACKGROUND

Multiple myeloma (MM) is a plasma cell malignancy. Despite present chemotherapy strategies which can extend survival, many patients succumb to this disease. Thus, there is a need to identify improved treatments. Venetoclax is clinically approved for the treatment of chronic lymphocytic leukemia (CLL) and small lymphocytic lymphoma (SLL). However, some patients experience tumor lysis syndrome (TLS) which is a serious side effect sometimes causing kidney failure and death. Thus, there is a need to identify therapeutic methods that avoid the development of TLS.

Richardson et al. report iron chelators as therapeutic agents for the treatment of cancer. Crit Rev Oncol Hematol, 2002, 42(3): 267-81.

Campanella et al. report iron increases the susceptibility of multiple myeloma cells to bortezomib. Haematologica, 2013, 98(6): 971-9.

Shanmugam et al. report methods of treating cancer with a combination of glucose modulators and BCL-2 inhibitors. US Pat Pub No 2016/0113925.

Guieze et al. report mitochondrial reprogramming underlies resistance to BCL-2 inhibition in lymphoid malignancies. Cancer Cell, 2019, 36, 369-384.

Bajpai et al. report electron transport chain activity is a predictor and target for venetoclax sensitivity in multiple myeloma. Nat Comm, 2020, 11, 1228.

References cited herein are not an admission of prior art.

### SUMMARY

Disclosed herein are methods of managing cancer treatments using an anticancer agent and iron, heme, iron complexes, and/or iron salts. In certain embodiments, this disclosure relates to methods of treating cancer comprising administering to a patient in need thereof an effective amount of a BCL-2 inhibitor in combination with iron, heme, iron complex, and/or iron salt. In certain embodiments, the cancer is a hematological malignancy. In certain embodiments, the iron complex is a heme iron complex. In certain embodiments, the iron, iron complex, and/or iron salt is heme containing iron, ferric ammonium citrate, ferrous sulfate, or ferumoxytol.

In certain embodiments, this disclosure relates to methods of treating cancer comprising administering to a patient in need thereof an effective amount of an anticancer agent, such as a BCL-2 inhibitor, in combination with iron, heme, iron complex, and/or iron salt thereof. In certain embodiments, administering to a patient in need thereof an effective amount of a BCL-2 inhibitor in combination with iron, iron complex, or iron salt is in combination with heme or other iron complexing agent.

In certain embodiments, this disclosure relates to methods of treating cancer comprising administering to a patient in need thereof an effective amount of an anticancer agent, such as a BCL-2 inhibitor, in combination with an inhibitors of the RAS-RAF-MEK-ERK pathway e.g., Raf inhibitors, such as vemurafenib and dabrafenib or MEK inhibitors such as binimetinib, cobimetinib, selumetinib, trametinib or ERK inhibitors such as ulixertinib, rineterkib, dordaviprone, honokiol, and in combination with iron, heme, iron complex, and/or iron salt thereof.

In certain embodiments, administering to a patient in need thereof an effective amount of a BCL-2 inhibitor in combination with iron, iron complex, or iron salt is in combination with heme containing iron or other iron complexing agent.

In certain embodiments, the BCL-2 inhibitor is venetoclax. In certain embodiments, the cancer is multiple myeloma. In certain embodiments, the patient is diagnosed with or without a t(11; 14) translocation.

In certain embodiments, the BCL-2 inhibitor is administered in combination with another anti-cancer agent, e.g., such as a MEK inhibitor. In certain embodiments, administration of the BCL-2 inhibitor and the anti-cancer agent/MEK inhibitor are in combination with iron or heme containing iron or other iron complexing agent.

In certain embodiments, the BCL-2 inhibitor is administered at a lower amount when compared to the amount normally clinically administered considering the weight of the patient.

In certain embodiments, this disclosure relates to methods of treating cancer comprising administering to a patient in need thereof an effective amount of a BCL-2 inhibitor in combination with a SLC7A11 inhibitor. In certain embodiments, the SLC7A11 inhibitor is erastin. In certain embodiments, the BCL-2 inhibitor is venetoclax. In certain embodiments, administering to a patient in need thereof an effective amount of a BCL-2 inhibitor and a SLC7A11 inhibitor is in combination with administering with iron, heme, iron complex, or iron salt. In certain embodiments, administering to a patient in need thereof an effective amount of a BCL-2 inhibitor, a SLC7A11 inhibitor, and iron, iron complex, or iron salt is in combination with heme containing iron or an alternative iron complexing agent. In certain embodiments, the cancer is multiple myeloma. In certain embodiments, the patient is diagnosed with or without a t(11; 14) translocation.

In certain embodiments, this disclosure relates to methods of diagnosing and treating a patient with cancer comprising diagnosing whether the patient has low levels of aminolevulinic acid (ALA) by obtaining a sample from the patient and detecting in the sample low levels of aminolevulinic acid (ALA) providing a diagnosis of low levels of aminolevulinic acid (ALA); and if the subject is diagnosed with low levels of aminolevulinic acid (ALA) administering to the patient an effective amount of a BCL-2 inhibitor in combination with iron, heme, iron complex, or iron salt as reported herein. In certain embodiments, the BCL-2 inhibitor is venetoclax. In certain embodiments, the cancer is multiple myeloma. In certain embodiments, the subject is diagnosed with or without a t(11; 14) translocation.

### BRIEF DESCRIPTION OF THE SEVERAL VIEWS OF THE DRAWINGS

FIG. 1 shows data indicating t(11; 14) demonstrates reduced heme biosynthesis: detected by assessing ALA incorporation in protoporphyrin.

FIG. 2 show data indicating supplementation with hemin not ALA (precursor for heme synthesis) reverses venetoclax sensitivity.

FIG. 3 shows data indicating a high dose of ALA targets venetoclax sensitive multiple myeloma MM.

FIG. 4 shows data indicating inhibition of MEK (MEKi) suppressed hemin mediated venetoclax resistance (VR).

FIG. 5 shows data indicating inhibiting heme biosynthesis promotes venetoclax (Ven) sensitivity in multiple myeloma (MM) patient samples.

FIG. 6 shows data indicating supplementation with hemin reverses venetoclax sensitivity in venetoclax sensitive acute myeloid leukemia (AML) cell lines.

FIG. 7A shows data indicating ferric ammonium citrate (FAC) supplementation sensitizes t(11; 14) cells to venetoclax (Ven). Cells were treated with 500 μM FAC up to 96 h and assessed for viability using CellTiter-Glo® 3D Cell Viability Assay.

FIG. 7B shows data indicating ferric ammonium citrate (FAC) supplementation sensitizes non t(11,14) MM cell lines.

FIG. 7C shows data from MM patient samples treated with FAC (250 μM and 500 μM) and venetoclax (0.001μM, 0.005 μM and 0.01 μM) to assess cell death/viability by Annexin V/FITC flow cytometric staining.

FIG. 8A shows data on the evaluation of mitochondrial reactive oxygen species (ROS) (MitoROS™) after 48 h of FAC supplementation in venetoclax sensitive (VS) cell lines.

FIG. 8B show data on the expression of iron homeostasis-related protein in whole cell lysates supplemented with 500 μM FAC for 72 h in indicated cell lines by immunoblotting using actin as a loading control. Indicated cell lines were treated with a ferroptotic inhibitor, ferrostatin (Fn-1).

FIG. 9A shows data where venetoclax sensitive (VS) multiple myeloma (MM) cell lines were pre-treated with 50 μM Hemin/5-ALA for 24 hours, followed by co-treatment with increasing doses of Ven for 24 hrs. Cell death/viability was assessed by Annexin V/DAPI flow cytometric staining.

FIG. 9B shows data indicating hemin restores MEK-ERK-mTORC1 signaling in VS MM. Whole cell lysates of indicated lines were treated with 50 μM hemin and/or 0.05 μM Ven for 18 h. Indicated VS cells were treated with 50 M hemin and 0.05 μM Ven and RAF, MEK, or ERK-inhibitors. Cell death/viability was evaluated by Annexin V/DAPI flow cytometric staining.

FIG. 9C show data indicating ferrochelatase (FECH) inhibition sensitizes both VR and VS MM to Ven VS cells treated with a FECH inhibitor (FECHi), SH-17023. MM patient samples treated with 25 M of FECH inhibitor and 50 μM Hemin with increasing doses of Ven for 24hrs.

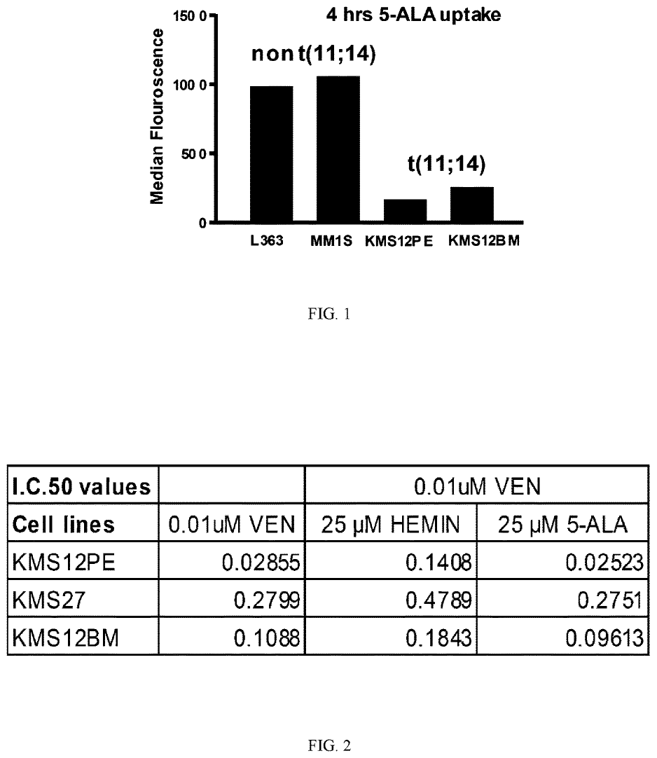

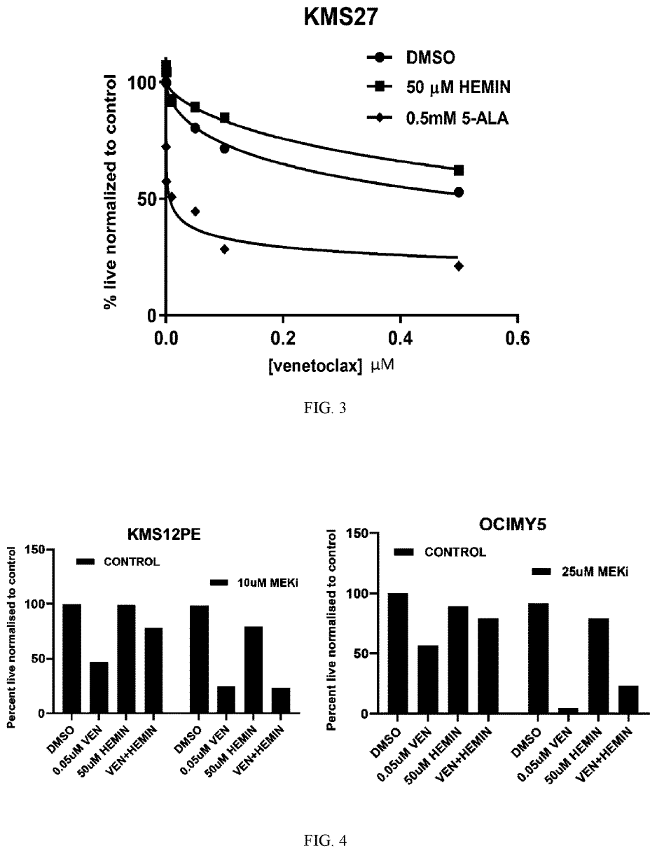

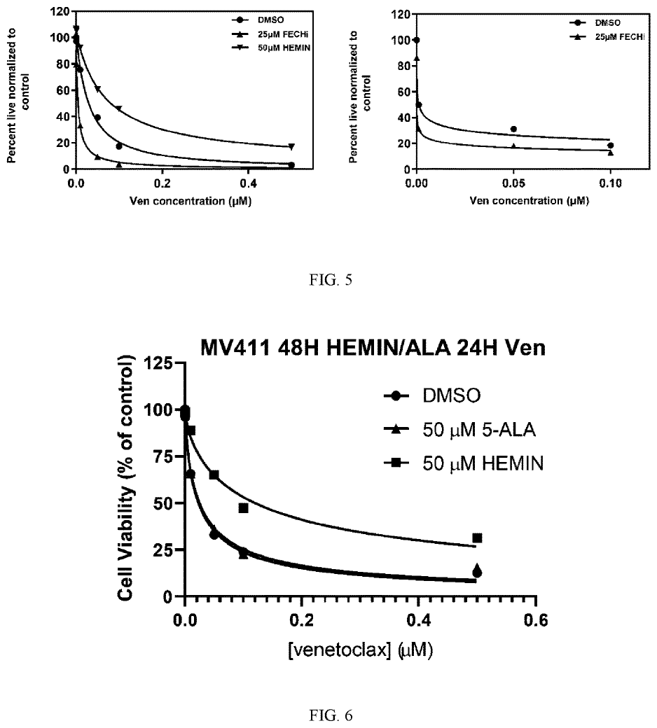

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims or as amended during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” is an example, and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art.

The terms “co-administration,” “administered in combination with,” and their grammatical equivalents, as used herein, encompass administration of two or more agents to subject so that both agents and/or their individual agents, complexes, or metabolites are present in the subject at the same time. Co-administration includes simultaneous administration in separate compositions, administration at different times in separate compositions, or administration in a composition in which both agents are present.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

Heme, a component of hemoglobin, is used in animals to transport oxygen. Heme is made up of ferric or ferrous iron ligated to a ring tetrapyrroles which gives blood its distinctive red color. In humans, the tetrapyrrole ring is derivatized with four methyl groups, two vinyl groups, and two propionate side chains. “Heme” refers to a chemical compound having the core tetrapyrrole ring structure of iron(2+) porphyrin-21,23-diide. Structural variants of heme are present in nature and well documented in the literature. Heme B (also known as protoporphyrin IX) has the chemical name iron(2+) 2,18-bis(2-carboxyethyl)-3,8,13,17-tetramethyl-7,12-divinylporphine-21,23-diide. Hemin refers to the [7,12-diethenyl-3,8,13,17-tetramethyl-21H,23H-porphine-2,18-dipropanoato(2-)-N21,N22,N23,N24] iron or alternative salt thereof, e.g., mono-chloride salt. In certain embodiments, the heme may be hemin, heme A, heme B, heme C, or heme O. Common heme variants are tetrapyrrole ring structures that comprises the follow substituents CH₂CH₂CO₂H, —CH(OH)CH₂farnesenyl, —CH═CH₂, —CH(cystein-S-yl)CH₃, —CH═O, —CH₃.

### Methods of Treating Cancer Using BCL-2 Inhibitors and Iron

Cancerous cells evade a self-destruction mechanism referred to as apoptosis. The BCL-2 family of proteins regulate apoptosis. Certain BH3-only proteins have the ability to activate effector proteins BAX and/or BAK which oligomerize and permeabilize the outer mitochondrial membrane releasing cytochrome c to activate subsequent steps of apoptosis resulting in cell death. Upregulation of antiapoptotic proteins BCL-2, MCL-1, or BCL-XL help cancer cells evade apoptosis. BCL-2 inhibitors interact with members of the BCL2 family of proteins resulting in antitumor effects. Although it is not intended that embodiments of this disclosure be limited by any particular mechanism, it is contemplated that BCL-2 inhibitors are effective in cancer treatments by reducing the production of anti-apoptotic proteins or blocking the anti-apoptotic mechanisms of tumor cells.

In certain embodiments, this disclosure relates to methods of treating cancer comprising administering to a patient in need thereof an effective amount of an anticancer agent, such as a BCL-2 inhibitor, in combination with iron, iron complex, and/or iron salt thereof. In certain embodiments, administering to a patient in need thereof an effective amount of a BCL-2 inhibitor in combination with iron, iron complex, or iron salt is in combination with heme containing iron or other iron complexing agent.

In certain embodiments, this disclosure relates to methods of treating cancer comprising administering to a patient in need thereof an effective amount of an anticancer agent, such as a BCL-2 inhibitor, in combination with inhibitors of the RAS-RAF-MEK-ERK pathway e.g., Raf inhibitor, such as vemurafenib and dabrafenib or a MEK inhibitor such as binimetinib, cobimetinib, selumetinib, trametinib, or a ERK inhibitor such as ulixertinib, rineterkib, dordaviprone, honokiol, and in combination with iron, heme, iron complex, and/or iron salt thereof specific.

In certain embodiments, the BCL-2 inhibitor is venetoclax. In certain embodiments, the cancer is multiple myeloma. In certain embodiments, the patient is diagnosed with or without a t(11; 14) translocation.

In certain embodiments, the BCL-2 inhibitor is administered in combination with another anti-cancer agent, e.g., such as a MEK inhibitor. In certain embodiments, administration of the BCL-2 inhibitor and the anti-cancer agent/MEK inhibitor are in combination with heme containing iron or other iron complexing agent.

In certain embodiments, the iron, iron complex, and/or iron salt is heme containing iron, ferric ammonium citrate, ferrous sulfate, or ferumoxytol.

In certain embodiments, the BCL-2 inhibitor is venetoclax in combination with iron, heme, iron complex, and/or iron salt thereof. In certain embodiments, the cancer is a hematological cancer such as multiple myeloma (MM), mantle cell lymphoma (MCL), chronic lymphocytic leukemia (CLL), or acute myeloid leukemia (AML). In certain embodiments, the patient is diagnosed with or without a t(11; 14) translocation.

In certain embodiments, this disclosure relates to methods of treating cancer comprising administering to a patient in need thereof an effective amount of a BCL-2 inhibitor in combination with a SLC7A11 inhibitor. In certain embodiments, the SLC7A11 inhibitor is erastin. In certain embodiments, the BCL-2 inhibitor is venetoclax. In certain embodiments, administering to a patient in need thereof an effective amount of a BCL-2 inhibitor and a SLC7A11 inhibitor is in combination with administering with heme, iron, iron complex, or iron salt. In certain embodiments, administering to a patient in need thereof an effective amount of a BCL-2 inhibitor and a SLC7A11 inhibitor is in combination with iron, heme, or other iron complexing agent. In certain embodiments, the cancer is multiple myeloma or other hematological malignancy. In certain embodiments, the patient is diagnosed with or without a t(11; 14) translocation. Ni et al. report translocation t(11; 14) (q13; q32) and genomic imbalances in multiple myeloma patients. Hematol Rep, 2012, 4(3): e19. Fluorescence in situ hybridization, PCR, and other techniques can be used for detecting t(11; 14)(q13; q32) translocation in patients. Cells with a t(11; 14)(q13; q32) translocation in the chromosomal DNA generally over-express cyclin D1.

In certain embodiments, this disclosure relates to methods of diagnosing and treating a patient with cancer comprising diagnosing whether the patient has low levels of aminolevulinic acid (ALA) by obtaining a sample from the patient and detecting in the sample low levels of aminolevulinic acid (ALA) providing a diagnosis of low levels of aminolevulinic acid (ALA); and if the subject is diagnosed with low levels of aminolevulinic acid (ALA) administering to the patient an effective amount of a BCL-2 inhibitor in combination with heme, iron, iron complex, and/or iron salt. In certain embodiments, the BCL-2 inhibitor is venetoclax. In certain embodiments, the cancer is multiple myeloma or other hematological malignancy. In certain embodiments, the subject is diagnosed with or without a t(11; 14) translocation.

In certain embodiments, the BCL-2 inhibitor is venetoclax, navitoclax, obatoclax, or oblimersen in combination with heme, iron, iron complex, and/or iron salt thereof. In certain embodiments, the patient is diagnosed with or without a t(11; 14) translocation. In certain embodiments, the cancer is a hematological malignancy such as multiple myeloma (MM), chronic lymphocytic leukemia (CLL), small lymphocytic lymphoma (SLL), or acute myeloid leukemia (AML).

In certain embodiments, the iron, heme, or other iron complexing agent and BCL-2 inhibitor is further administered in combination with fludarabine, dexamethasone, bortezomib, azacytidine, bendamustine, obinutuzumab, chlorambucil, rituximab, cytosine arabinoside, or a hypomethylating agent. In certain embodiments, the hypomethylating agent is azacitidine (5-azacytidine) or decitabine (5-aza-2′-deoxycytidine).

In certain embodiments, the BCL-2 inhibitor is administered in combination with a MEK inhibitor such as trametinib, cobimetinib, binimetinib, and mirdametinib. In certain embodiments, administration of the BCL-2 inhibitor and the MEK inhibitor are in combination with iron, heme, or other iron complexing agent.

In certain embodiments, the BCL-2 inhibitor is administered in combination with a BRAF inhibitor such as vemurafenib, dabrafenib, or encorafenib. In certain embodiments, administration of the BCL-2 inhibitor and the BRAF inhibitor are in combination with iron, heme, or other iron complexing agent.

In certain embodiments, the cancer to be treated is a solid tumor, cellular malignancy, or hematological malignancy. In certain embodiments, the cancer is a hematological cancer such as myeloma, multiple myeloma (MM), leukemia, or lymphoma. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages, and mast cells; the lymphoid cell line produces B, T, NK, and plasma cells. Lymphomas, lymphocytic leukemias, and myeloma are from the lymphoid line, while acute and chronic myelogenous leukemia, myelodysplastic syndromes and myeloproliferative diseases are myeloid in origin.

In certain embodiments, the cancer is lung cancer, non-small cell lung cancer, small cell lung cancer, bronchus cancer, tumors originating in the lung parenchyma or within the bronchi, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, nasopharyngeal carcinoma, or ependymoma,.

Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genitourinary organs and, more particularly, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

In certain embodiments, the BCL-2 inhibitor is administered at a lower amount when compared to the amount normally clinically administered considering the weight of the patient.

In certain embodiments, this disclosure relates to methods of treating cancer comprising administering to a patient in need thereof an effective amount of iron, heme, or other iron complexing agent and BCL-2 inhibitor in an amount of 5 mg daily for two or more weeks. In certain embodiments, this disclosure relates to methods of treating cancer comprising administering to a patient in need thereof an effective amount of iron, heme, or other iron complexing agent and BCL-2 inhibitor in an amount of 5 mg daily for three or more weeks. In certain embodiments, this disclosure relates to methods of treating cancer comprising administering to a patient in need thereof an effective amount of iron, heme, or other iron complexing agent and BCL-2 inhibitor in an amount of 5 mg daily for four or more weeks.

In certain embodiments, this disclosure relates to methods of treating cancer comprising administering to a patient in need thereof an effective amount of iron, heme, or other iron complexing agent and BCL-2 inhibitor in an amount of 10 mg daily for two or more weeks. In certain embodiments, this disclosure relates to methods of treating cancer comprising administering to a patient in need thereof an effective amount of iron, heme, or other iron complexing agent and venetoclax in an amount of 10 mg daily for three or more weeks. In certain embodiments, this disclosure relates to methods of treating cancer comprising administering to a patient in need thereof an effective amount of iron, heme, or other iron complexing agent and BCL-2 inhibitor in an amount of 10 mg daily for four or more weeks.

In certain embodiments, this disclosure relates to methods of treating cancer comprising administering to a patient in need thereof an effective amount of iron, heme, or other iron complexing agent and BCL-2 inhibitor in an amount of 20 mg daily for two or more weeks. In certain embodiments, this disclosure relates to methods of treating cancer comprising administering to a patient in need thereof an effective amount of iron, heme, or other iron complexing agent and BCL-2 inhibitor in an amount of 20 mg daily for three or more weeks. In certain embodiments, this disclosure relates to methods of treating cancer comprising administering to a patient in need thereof an effective amount of iron, heme, or other iron complexing agent and BCL-2 inhibitor in an amount of 20 mg daily for four or more weeks.

In certain embodiments, this disclosure relates to methods of treating cancer comprising administering to a patient in need thereof an effective amount of iron, heme, or other iron complexing agent and BCL-2 inhibitor in an amount of 40 mg daily for two or more weeks. In certain embodiments, this disclosure relates to methods of treating cancer comprising administering to a patient in need thereof an effective amount of iron, heme, or other iron complexing agent and BCL-2 inhibitor in an amount of 40 mg daily for three or more weeks. In certain embodiments, this disclosure relates to methods of treating cancer comprising administering to a patient in need thereof an effective amount of iron, heme, or other iron complexing agent and BCL-2 inhibitor in an amount of 40 mg daily for four or more weeks.

In certain embodiments, this disclosure relates to methods of treating cancer comprising administering to a patient in need thereof an effective amount of iron, heme, or other iron complexing agent and venetoclax in an amount of 5 mg daily for two or more weeks. In certain embodiments, this disclosure relates to methods of treating cancer comprising administering to a patient in need thereof an effective amount of iron, heme, or other iron complexing agent and venetoclax in an amount of 5 mg daily for three or more weeks. In certain embodiments, this disclosure relates to methods of treating cancer comprising administering to a patient in need thereof an effective amount of iron, heme, or other iron complexing agent and venetoclax in an amount of 5 mg daily for four or more weeks.

In certain embodiments, this disclosure relates to methods of treating cancer comprising administering to a patient in need thereof an effective amount of iron, heme, or other iron complexing agent and venetoclax in an amount of 10 mg daily for two or more weeks. In certain embodiments, this disclosure relates to methods of treating cancer comprising administering to a patient in need thereof an effective amount of iron, heme, or other iron complexing agent and venetoclax in an amount of 10 mg daily for three or more weeks. In certain embodiments, this disclosure relates to methods of treating cancer comprising administering to a patient in need thereof an effective amount of iron, heme, or other iron complexing agent and venetoclax in an amount of 10 mg daily for four or more weeks.

In certain embodiments, this disclosure relates to methods of treating cancer comprising administering to a patient in need thereof an effective amount of iron, heme, or other iron complexing agent and venetoclax in an amount of 20 mg daily for two or more weeks. In certain embodiments, this disclosure relates to methods of treating cancer comprising administering to a patient in need thereof an effective amount of iron, heme, or other iron complexing agent and venetoclax in an amount of 20 mg daily for three or more weeks. In certain embodiments, this disclosure relates to methods of treating cancer comprising administering to a patient in need thereof an effective amount of iron, heme, or other iron complexing agent and venetoclax in an amount of 20 mg daily for four or more weeks.

In certain embodiments, this disclosure relates to methods of treating cancer comprising administering to a patient in need thereof an effective amount of iron, heme, or other iron complexing agent and venetoclax in an amount of 40 mg daily for two or more weeks. In certain embodiments, this disclosure relates to methods of treating cancer comprising administering to a patient in need thereof an effective amount of iron, heme, or other iron complexing agent and venetoclax in an amount of 40 mg daily for three or more weeks. In certain embodiments, this disclosure relates to methods of treating cancer comprising administering to a patient in need thereof an effective amount of iron, heme, or other iron complexing agent and venetoclax in an amount of 40 mg daily for four or more weeks.

In certain embodiments, this disclosure relates to methods of treating multiple myeloma (MM) comprising administering to a patient in need thereof an effective amount of iron, heme, or other iron complexing agent and venetoclax in an amount of 5 mg daily for two or more weeks. In certain embodiments, this disclosure relates to methods of treating multiple myeloma (MM), comprising administering to a patient in need thereof an effective amount of iron, heme, or other iron complexing agent and venetoclax in an amount of 5 mg daily for three or more weeks. In certain embodiments, this disclosure relates to methods of treating multiple myeloma (MM), comprising administering to a patient in need thereof an effective amount of iron, heme, or other iron complexing agent and venetoclax in an amount of 5 mg daily for four or more weeks.

In certain embodiments, this disclosure relates to methods of treating multiple myeloma (MM) comprising administering to a patient in need thereof an effective amount of iron, heme, or other iron complexing agent and venetoclax in an amount of 10 mg daily for two or more weeks. In certain embodiments, this disclosure relates to methods of treating multiple myeloma (MM) comprising administering to a patient in need thereof an effective amount of iron, heme, or other iron complexing agent and venetoclax in an amount of 10 mg daily for three or more weeks. In certain embodiments, this disclosure relates to methods of treating multiple myeloma (MM) comprising administering to a patient in need thereof an effective amount of iron, heme, or other iron complexing agent and venetoclax in an amount of 10 mg daily for four or more weeks.

In certain embodiments, this disclosure relates to methods of treating multiple myeloma (MM), comprising administering to a patient in need thereof an effective amount of iron, heme, or other iron complexing agent and venetoclax in an amount of 20 mg daily for two or more weeks. In certain embodiments, this disclosure relates to methods of treating multiple myeloma comprising administering to a patient in need thereof an effective amount of iron, heme, or other iron complexing agent and venetoclax in an amount of 20 mg daily for three or more weeks. In certain embodiments, this disclosure relates to methods of treating multiple myeloma (MM) comprising administering to a patient in need thereof an effective amount of iron, heme, or other iron complexing agent and venetoclax in an amount of 20 mg daily for four or more weeks.

In certain embodiments, this disclosure relates to methods of treating multiple myeloma (MM) comprising administering to a patient in need thereof an effective amount of iron, heme, or other iron complexing agent and venetoclax in an amount of 40 mg daily for two or more weeks. In certain embodiments, this disclosure relates to methods of treating multiple myeloma (MM) comprising administering to a patient in need thereof an effective amount of iron, heme, or other iron complexing agent and venetoclax in an amount of 40 mg daily for three or more weeks. In certain embodiments, this disclosure relates to methods of treating multiple myeloma (MM) comprising administering to a patient in need thereof an effective amount of iron, heme, or other iron complexing agent and venetoclax in an amount of 40 mg daily for four or more weeks.

In certain embodiments, this disclosure relates to methods of treating chronic lymphocytic leukemia (CLL) comprising administering to a patient in need thereof an effective amount of iron, heme, or other iron complexing agent and venetoclax in an amount of 5 mg daily for two or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 5 mg daily for three or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 5 mg daily for four or more weeks.

In certain embodiments, this disclosure relates to methods of treating chronic lymphocytic leukemia (CLL) comprising administering to a patient in need thereof an effective amount of iron, heme, or other iron complexing agent and venetoclax in an amount of 10 mg daily for two or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 10 mg daily for three or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 10 mg daily for four or more weeks.

In certain embodiments, this disclosure relates to methods of treating chronic lymphocytic leukemia (CLL) comprising administering to a patient in need thereof an effective amount of iron, heme, or other iron complexing agent and venetoclax in an amount of 20 mg daily for two or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 20 mg daily for three or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 20 mg daily for four or more weeks.

In certain embodiments, this disclosure relates to methods of treating chronic lymphocytic leukemia (CLL) comprising administering to a patient in need thereof an effective amount of iron, heme, or other iron complexing agent and venetoclax in an amount of 40 mg daily for two or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 40 mg daily for three or more weeks. In certain embodiments, this disclosure relates to methods of treating chronic lymphocytic leukemia (CLL) comprising administering to a patient in need thereof an effective amount of iron, heme, or other iron complexing agent and venetoclax in an amount of 40 mg daily for four or more weeks.

In certain embodiments, this disclosure relates to methods of treating multiple small lymphocytic lymphoma (SLL) comprising administering to a patient in need thereof an effective amount of iron, heme, or other iron complexing agent and venetoclax in an amount of 5 mg daily for two or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 5 mg daily for three or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 5 mg daily for four or more weeks.

In certain embodiments, this disclosure relates to methods of treating small lymphocytic lymphoma (SLL) comprising administering to a patient in need thereof an effective amount of iron, heme, or other iron complexing agent and venetoclax in an amount of 10 mg daily for two or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 10 mg daily for three or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 10 mg daily for four or more weeks.

In certain embodiments, this disclosure relates to methods of treating small lymphocytic lymphoma (SLL) comprising administering to a patient in need thereof an effective amount of iron, heme, or other iron complexing agent and venetoclax in an amount of 20 mg daily for two or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 20 mg daily for three or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 20 mg daily for four or more weeks.

In certain embodiments, this disclosure relates to methods of treating small lymphocytic lymphoma (SLL) comprising administering to a patient in need thereof an effective amount of iron, heme, or other iron complexing agent and venetoclax in an amount of 40 mg daily for two or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 40 mg daily for three or more weeks. In certain embodiments, this disclosure relates to methods of treating small lymphocytic lymphoma (SLL) comprising administering to a patient in need thereof an effective amount of iron, heme, or other iron complexing agent and venetoclax in an amount of 40 mg daily for four or more weeks.

In certain embodiments, this disclosure relates to methods of treating acute myeloid leukemia (AML) comprising administering to a patient in need thereof an effective amount of iron, heme, or other iron complexing agent and venetoclax in an amount of 5 mg daily for two or more weeks. In certain embodiments, this disclosure relates to methods of treating acute myeloid leukemia (AML) comprising administering to a patient in need thereof an effective amount of iron, heme, or other iron complexing agent and venetoclax in an amount of 5 mg daily for three or more weeks. In certain embodiments, this disclosure relates to methods of treating acute myeloid leukemia (AML)comprising administering to a patient in need thereof an effective amount of iron, heme, or other iron complexing agent and venetoclax in an amount of 5 mg daily for four or more weeks.

In certain embodiments, this disclosure relates to methods of treating acute myeloid leukemia (AML) comprising administering to a patient in need thereof an effective amount of iron, heme, or other iron complexing agent and venetoclax in an amount of 10 mg daily for two or more weeks. In certain embodiments, this disclosure relates to methods of treating acute myeloid leukemia (AML) comprising administering to a patient in need thereof an effective amount of iron, heme, or other iron complexing agent and venetoclax in an amount of 10 mg daily for three or more weeks. In certain embodiments, this disclosure relates to methods of treating acute myeloid leukemia (AML)comprising administering to a patient in need thereof an effective amount of iron, heme, or other iron complexing agent and venetoclax in an amount of 10 mg daily for four or more weeks.

In certain embodiments, this disclosure relates to methods of treating acute myeloid leukemia (AML) comprising administering to a patient in need thereof an effective amount of iron, heme, or other iron complexing agent and venetoclax in an amount of 20 mg daily for two or more weeks. In certain embodiments, this disclosure relates to methods of treating acute myeloid leukemia (AML) comprising administering to a patient in need thereof an effective amount of iron, heme, or other iron complexing agent and venetoclax in an amount of 20 mg daily for three or more weeks. In certain embodiments, this disclosure relates to methods of treating acute myeloid leukemia (AML) comprising administering to a patient in need thereof an effective amount of iron, heme, or other iron complexing agent and venetoclax in an amount of 20 mg daily for four or more weeks.

In certain embodiments, this disclosure relates to methods of treating acute myeloid leukemia (AML) comprising administering to a patient in need thereof an effective amount of iron, heme, or other iron complexing agent and venetoclax in an amount of 40 mg daily for two or more weeks. In certain embodiments, this disclosure relates to methods of treating acute myeloid leukemia (AML) comprising administering to a patient in need thereof an effective amount of iron, heme, or other iron complexing agent and venetoclax in an amount of 40 mg daily for three or more weeks. In certain embodiments, this disclosure relates to methods of treating acute myeloid leukemia (AML) comprising administering to a patient in need thereof an effective amount of iron, heme, or other iron complexing agent and venetoclax in an amount of 40 mg daily for four or more weeks.

In certain embodiments, this disclosure relates to methods of treating cancer comprising administering to a patient in need thereof an effective amount of iron, heme, or other iron complexing agent and navitoclax in an amount of 5 mg daily for two or more weeks. In certain embodiments, this disclosure relates to methods of treating cancer comprising administering to a patient in need thereof an effective amount of iron, heme, or other iron complexing agent and navitoclax in an amount of 5 mg daily for three or more weeks. In certain embodiments, this disclosure relates to methods of treating cancer comprising administering to a patient in need thereof an effective amount of iron, heme, or other iron complexing agent and navitoclax in an amount of 5 mg daily for four or more weeks.

In certain embodiments, this disclosure relates to methods of treating cancer comprising administering to a patient in need thereof an effective amount of iron, heme, or other iron complexing agent and navitoclax in an amount of 10 mg daily for two or more weeks. In certain embodiments, this disclosure relates to methods of treating cancer comprising administering to a patient in need thereof an effective amount of iron, heme, or other iron complexing agent and navitoclax in an amount of 10 mg daily for three or more weeks. In certain embodiments, this disclosure relates to methods of treating cancer comprising administering to a patient in need thereof an effective amount of iron, heme, or other iron complexing agent and navitoclax in an amount of 10 mg daily for four or more weeks.

In certain embodiments, this disclosure relates to methods of treating cancer comprising administering to a patient in need thereof an effective amount of iron, heme, or other iron complexing agent and navitoclax in an amount of 20 mg daily for two or more weeks. In certain embodiments, this disclosure relates to methods of treating cancer comprising administering to a patient in need thereof an effective amount of iron, heme, or other iron complexing agent and navitoclax in an amount of 20 mg daily for three or more weeks. In certain embodiments, this disclosure relates to methods of treating cancer comprising administering to a patient in need thereof an effective amount of iron, heme, or other iron complexing agent and navitoclax in an amount of 20 mg daily for four or more weeks.

In certain embodiments, this disclosure relates to methods of treating cancer comprising administering to a patient in need thereof an effective amount of iron, heme, or other iron complexing agent and navitoclax in an amount of 40 mg daily for two or more weeks. In certain embodiments, this disclosure relates to methods of treating cancer comprising administering to a patient in need thereof an effective amount of iron, heme, or other iron complexing agent and navitoclax in an amount of 40 mg daily for three or more weeks. In certain embodiments, this disclosure relates to methods of treating cancer comprising administering to a patient in need thereof an effective amount of iron, heme, or other iron complexing agent and navitoclax in an amount of 40 mg daily for four or more weeks.

In certain embodiments, this disclosure relates to methods of treating multiple myeloma (MM) comprising administering to a patient in need thereof an effective amount of iron, heme, or other iron complexing agent and navitoclax in an amount of 5 mg daily for two or more weeks. In certain embodiments, this disclosure relates to methods of treating multiple myeloma (MM), comprising administering to a patient in need thereof an effective amount of iron, heme, or other iron complexing agent and navitoclax in an amount of 5 mg daily for three or more weeks. In certain embodiments, this disclosure relates to methods of treating multiple myeloma (MM), comprising administering to a patient in need thereof an effective amount of iron, heme, or other iron complexing agent and navitoclax in an amount of 5 mg daily for four or more weeks.

In certain embodiments, this disclosure relates to methods of treating multiple myeloma (MM) comprising administering to a patient in need thereof an effective amount of iron, heme, or other iron complexing agent and navitoclax in an amount of 10 mg daily for two or more weeks. In certain embodiments, this disclosure relates to methods of treating multiple myeloma (MM) comprising administering to a patient in need thereof an effective amount of iron, heme, or other iron complexing agent and navitoclax in an amount of 10 mg daily for three or more weeks. In certain embodiments, this disclosure relates to methods of treating multiple myeloma (MM) comprising administering to a patient in need thereof an effective amount of iron, heme, or other iron complexing agent and navitoclax in an amount of 10 mg daily for four or more weeks.

In certain embodiments, this disclosure relates to methods of treating multiple myeloma (MM), comprising administering to a patient in need thereof an effective amount of iron, heme, or other iron complexing agent and navitoclax in an amount of 20 mg daily for two or more weeks. In certain embodiments, this disclosure relates to methods of treating multiple myeloma comprising administering to a patient in need thereof an effective amount of iron, heme, or other iron complexing agent and navitoclax in an amount of 20 mg daily for three or more weeks. In certain embodiments, this disclosure relates to methods of treating multiple myeloma (MM) comprising administering to a patient in need thereof an effective amount of iron, heme, or other iron complexing agent and navitoclax in an amount of 20 mg daily for four or more weeks.

In certain embodiments, this disclosure relates to methods of treating multiple myeloma (MM) comprising administering to a patient in need thereof an effective amount of iron, heme, or other iron complexing agent and navitoclax in an amount of 40 mg daily for two or more weeks. In certain embodiments, this disclosure relates to methods of treating multiple myeloma (MM) comprising administering to a patient in need thereof an effective amount of iron, heme, or other iron complexing agent and navitoclax in an amount of 40 mg daily for three or more weeks. In certain embodiments, this disclosure relates to methods of treating multiple myeloma (MM) comprising administering to a patient in need thereof an effective amount of iron, heme, or other iron complexing agent and navitoclax in an amount of 40 mg daily for four or more weeks.

In certain embodiments, this disclosure relates to methods of treating chronic lymphocytic leukemia (CLL) comprising administering to a patient in need thereof an effective amount of iron, heme, or other iron complexing agent and navitoclax in an amount of 5 mg daily for two or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 5 mg daily for three or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 5 mg daily for four or more weeks.

In certain embodiments, this disclosure relates to methods of treating chronic lymphocytic leukemia (CLL) comprising administering to a patient in need thereof an effective amount of iron, heme, or other iron complexing agent and navitoclax in an amount of 10 mg daily for two or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 10 mg daily for three or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 10 mg daily for four or more weeks.

In certain embodiments, this disclosure relates to methods of treating chronic lymphocytic leukemia (CLL) comprising administering to a patient in need thereof an effective amount of iron, heme, or other iron complexing agent and navitoclax in an amount of 20 mg daily for two or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 20 mg daily for three or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 20 mg daily for four or more weeks.

In certain embodiments, this disclosure relates to methods of treating chronic lymphocytic leukemia (CLL) comprising administering to a patient in need thereof an effective amount of iron, heme, or other iron complexing agent and navitoclax in an amount of 40 mg daily for two or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 40 mg daily for three or more weeks. In certain embodiments, this disclosure relates to methods of treating chronic lymphocytic leukemia (CLL) comprising administering to a patient in need thereof an effective amount of iron, heme, or other iron complexing agent and navitoclax in an amount of 40 mg daily for four or more weeks.

In certain embodiments, this disclosure relates to methods of treating multiple small lymphocytic lymphoma (SLL) comprising administering to a patient in need thereof an effective amount of iron, heme, or other iron complexing agent and navitoclax in an amount of 5 mg daily for two or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 5 mg daily for three or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 5 mg daily for four or more weeks.

In certain embodiments, this disclosure relates to methods of treating small lymphocytic lymphoma (SLL) comprising administering to a patient in need thereof an effective amount of iron, heme, or other iron complexing agent and navitoclax in an amount of 10 mg daily for two or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 10 mg daily for three or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 10 mg daily for four or more weeks.

In certain embodiments, this disclosure relates to methods of treating small lymphocytic lymphoma (SLL) comprising administering to a patient in need thereof an effective amount of iron, heme, or other iron complexing agent and navitoclax in an amount of 20 mg daily for two or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 20 mg daily for three or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 20 mg daily for four or more weeks.

In certain embodiments, this disclosure relates to methods of treating small lymphocytic lymphoma (SLL) comprising administering to a patient in need thereof an effective amount of iron, heme, or other iron complexing agent and navitoclax in an amount of 40 mg daily for two or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 40 mg daily for three or more weeks. In certain embodiments, this disclosure relates to methods of treating small lymphocytic lymphoma (SLL) comprising administering to a patient in need thereof an effective amount of iron, heme, or other iron complexing agent and navitoclax in an amount of 40 mg daily for four or more weeks.

In certain embodiments, this disclosure relates to methods of treating acute myeloid leukemia (AML) comprising administering to a patient in need thereof an effective amount of iron, heme, or other iron complexing agent and navitoclax in an amount of 5 mg daily for two or more weeks. In certain embodiments, this disclosure relates to methods of treating acute myeloid leukemia (AML) comprising administering to a patient in need thereof an effective amount of iron, heme, or other iron complexing agent and navitoclax in an amount of 5 mg daily for three or more weeks. In certain embodiments, this disclosure relates to methods of treating acute myeloid leukemia (AML)comprising administering to a patient in need thereof an effective amount of iron, heme, or other iron complexing agent and navitoclax in an amount of 5 mg daily for four or more weeks.

In certain embodiments, this disclosure relates to methods of treating acute myeloid leukemia (AML) comprising administering to a patient in need thereof an effective amount of iron, heme, or other iron complexing agent and navitoclax in an amount of 10 mg daily for two or more weeks. In certain embodiments, this disclosure relates to methods of treating acute myeloid leukemia (AML) comprising administering to a patient in need thereof an effective amount of iron, heme, or other iron complexing agent and navitoclax in an amount of 10 mg daily for three or more weeks. In certain embodiments, this disclosure relates to methods of treating acute myeloid leukemia (AML)comprising administering to a patient in need thereof an effective amount of iron, heme, or other iron complexing agent and navitoclax in an amount of 10 mg daily for four or more weeks.

In certain embodiments, this disclosure relates to methods of treating acute myeloid leukemia (AML) comprising administering to a patient in need thereof an effective amount of iron, heme, or other iron complexing agent and navitoclax in an amount of 20 mg daily for two or more weeks. In certain embodiments, this disclosure relates to methods of treating acute myeloid leukemia (AML) comprising administering to a patient in need thereof an effective amount of iron, heme, or other iron complexing agent and navitoclax in an amount of 20 mg daily for three or more weeks. In certain embodiments, this disclosure relates to methods of treating acute myeloid leukemia (AML) comprising administering to a patient in need thereof an effective amount of iron, heme, or other iron complexing agent and navitoclax in an amount of 20 mg daily for four or more weeks.

In certain embodiments, this disclosure relates to methods of treating acute myeloid leukemia (AML) comprising administering to a patient in need thereof an effective amount of iron, heme, or other iron complexing agent and navitoclax in an amount of 40 mg daily for two or more weeks. In certain embodiments, this disclosure relates to methods of treating acute myeloid leukemia (AML) comprising administering to a patient in need thereof an effective amount of iron, heme, or other iron complexing agent and navitoclax in an amount of 40 mg daily for three or more weeks. In certain embodiments, this disclosure relates to methods of treating acute myeloid leukemia (AML) comprising administering to a patient in need thereof an effective amount of iron, heme, or other iron complexing agent and navitoclax in an amount of 40 mg daily for four or more weeks.

In certain embodiments, this disclosure relates to methods of treating cancer comprising administering to a patient in need thereof an effective amount of iron, heme, or other iron complexing agent and obatoclax in an amount of 5 mg daily for two or more weeks. In certain embodiments, this disclosure relates to methods of treating cancer comprising administering to a patient in need thereof an effective amount of iron, heme, or other iron complexing agent and obatoclax in an amount of 5 mg daily for three or more weeks. In certain embodiments, this disclosure relates to methods of treating cancer comprising administering to a patient in need thereof an effective amount of iron, heme, or other iron complexing agent and obatoclax in an amount of 5 mg daily for four or more weeks.

In certain embodiments, this disclosure relates to methods of treating cancer comprising administering to a patient in need thereof an effective amount of iron, heme, or other iron complexing agent and obatoclax in an amount of 10 mg daily for two or more weeks. In certain embodiments, this disclosure relates to methods of treating cancer comprising administering to a patient in need thereof an effective amount of iron, heme, or other iron complexing agent and obatoclax in an amount of 10 mg daily for three or more weeks. In certain embodiments, this disclosure relates to methods of treating cancer comprising administering to a patient in need thereof an effective amount of iron, heme, or other iron complexing agent and obatoclax in an amount of 10 mg daily for four or more weeks.

In certain embodiments, this disclosure relates to methods of treating cancer comprising administering to a patient in need thereof an effective amount of iron, heme, or other iron complexing agent and obatoclax in an amount of 20 mg daily for two or more weeks. In certain embodiments, this disclosure relates to methods of treating cancer comprising administering to a patient in need thereof an effective amount of iron, heme, or other iron complexing agent and obatoclax in an amount of 20 mg daily for three or more weeks. In certain embodiments, this disclosure relates to methods of treating cancer comprising administering to a patient in need thereof an effective amount of iron, heme, or other iron complexing agent and obatoclax in an amount of 20 mg daily for four or more weeks.

In certain embodiments, this disclosure relates to methods of treating cancer comprising administering to a patient in need thereof an effective amount of iron, heme, or other iron complexing agent and obatoclax in an amount of 40 mg daily for two or more weeks. In certain embodiments, this disclosure relates to methods of treating cancer comprising administering to a patient in need thereof an effective amount of iron, heme, or other iron complexing agent and obatoclax in an amount of 40 mg daily for three or more weeks. In certain embodiments, this disclosure relates to methods of treating cancer comprising administering to a patient in need thereof an effective amount of iron, heme, or other iron complexing agent and obatoclax in an amount of 40 mg daily for four or more weeks.

In certain embodiments, this disclosure relates to methods of treating multiple myeloma (MM) comprising administering to a patient in need thereof an effective amount of iron, heme, or other iron complexing agent and obatoclax in an amount of 5 mg daily for two or more weeks. In certain embodiments, this disclosure relates to methods of treating multiple myeloma (MM), comprising administering to a patient in need thereof an effective amount of iron, heme, or other iron complexing agent and obatoclax in an amount of 5 mg daily for three or more weeks. In certain embodiments, this disclosure relates to methods of treating multiple myeloma (MM), comprising administering to a patient in need thereof an effective amount of iron, heme, or other iron complexing agent and obatoclax in an amount of 5 mg daily for four or more weeks.

In certain embodiments, this disclosure relates to methods of treating multiple myeloma (MM) comprising administering to a patient in need thereof an effective amount of iron, heme, or other iron complexing agent and obatoclax in an amount of 10 mg daily for two or more weeks. In certain embodiments, this disclosure relates to methods of treating multiple myeloma (MM) comprising administering to a patient in need thereof an effective amount of iron, heme, or other iron complexing agent and obatoclax in an amount of 10 mg daily for three or more weeks. In certain embodiments, this disclosure relates to methods of treating multiple myeloma (MM) comprising administering to a patient in need thereof an effective amount of iron, heme, or other iron complexing agent and obatoclax in an amount of 10 mg daily for four or more weeks.

In certain embodiments, this disclosure relates to methods of treating multiple myeloma (MM), comprising administering to a patient in need thereof an effective amount of iron, heme, or other iron complexing agent and obatoclax in an amount of 20 mg daily for two or more weeks. In certain embodiments, this disclosure relates to methods of treating multiple myeloma comprising administering to a patient in need thereof an effective amount of iron, heme, or other iron complexing agent and obatoclax in an amount of 20 mg daily for three or more weeks. In certain embodiments, this disclosure relates to methods of treating multiple myeloma (MM) comprising administering to a patient in need thereof an effective amount of iron, heme, or other iron complexing agent and obatoclax in an amount of 20 mg daily for four or more weeks.

In certain embodiments, this disclosure relates to methods of treating multiple myeloma (MM) comprising administering to a patient in need thereof an effective amount of iron, heme, or other iron complexing agent and obatoclax in an amount of 40 mg daily for two or more weeks. In certain embodiments, this disclosure relates to methods of treating multiple myeloma (MM) comprising administering to a patient in need thereof an effective amount of iron, heme, or other iron complexing agent and obatoclax in an amount of 40 mg daily for three or more weeks. In certain embodiments, this disclosure relates to methods of treating multiple myeloma (MM) comprising administering to a patient in need thereof an effective amount of iron, heme, or other iron complexing agent and obatoclax in an amount of 40 mg daily for four or more weeks.

In certain embodiments, this disclosure relates to methods of treating chronic lymphocytic leukemia (CLL) comprising administering to a patient in need thereof an effective amount of iron, heme, or other iron complexing agent and obatoclax in an amount of 5 mg daily for two or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 5 mg daily for three or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 5 mg daily for four or more weeks.

In certain embodiments, this disclosure relates to methods of treating chronic lymphocytic leukemia (CLL) comprising administering to a patient in need thereof an effective amount of iron, heme, or other iron complexing agent and obatoclax in an amount of 10 mg daily for two or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 10 mg daily for three or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 10 mg daily for four or more weeks.

In certain embodiments, this disclosure relates to methods of treating chronic lymphocytic leukemia (CLL) comprising administering to a patient in need thereof an effective amount of iron, heme, or other iron complexing agent and obatoclax in an amount of 20 mg daily for two or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 20 mg daily for three or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 20 mg daily for four or more weeks.

In certain embodiments, this disclosure relates to methods of treating chronic lymphocytic leukemia (CLL) comprising administering to a patient in need thereof an effective amount of iron, heme, or other iron complexing agent and obatoclax in an amount of 40 mg daily for two or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 40 mg daily for three or more weeks. In certain embodiments, this disclosure relates to methods of treating chronic lymphocytic leukemia (CLL) comprising administering to a patient in need thereof an effective amount of iron, heme, or other iron complexing agent and obatoclax in an amount of 40 mg daily for four or more weeks.

In certain embodiments, this disclosure relates to methods of treating multiple small lymphocytic lymphoma (SLL) comprising administering to a patient in need thereof an effective amount of iron, heme, or other iron complexing agent and obatoclax in an amount of 5 mg daily for two or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 5 mg daily for three or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 5 mg daily for four or more weeks.

In certain embodiments, this disclosure relates to methods of treating small lymphocytic lymphoma (SLL) comprising administering to a patient in need thereof an effective amount of iron, heme, or other iron complexing agent and obatoclax in an amount of 10 mg daily for two or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 10 mg daily for three or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 10 mg daily for four or more weeks.

In certain embodiments, this disclosure relates to methods of treating small lymphocytic lymphoma (SLL) comprising administering to a patient in need thereof an effective amount of iron, heme, or other iron complexing agent and obatoclax in an amount of 20 mg daily for two or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 20 mg daily for three or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 20 mg daily for four or more weeks.

In certain embodiments, this disclosure relates to methods of treating small lymphocytic lymphoma (SLL) comprising administering to a patient in need thereof an effective amount of iron, heme, or other iron complexing agent and obatoclax in an amount of 40 mg daily for two or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 40 mg daily for three or more weeks. In certain embodiments, this disclosure relates to methods of treating small lymphocytic lymphoma (SLL) comprising administering to a patient in need thereof an effective amount of iron, heme, or other iron complexing agent and obatoclax in an amount of 40 mg daily for four or more weeks.

In certain embodiments, this disclosure relates to methods of treating acute myeloid leukemia (AML) comprising administering to a patient in need thereof an effective amount of iron, heme, or other iron complexing agent and obatoclax in an amount of 5 mg daily for two or more weeks. In certain embodiments, this disclosure relates to methods of treating acute myeloid leukemia (AML) comprising administering to a patient in need thereof an effective amount of iron, heme, or other iron complexing agent and obatoclax in an amount of 5 mg daily for three or more weeks. In certain embodiments, this disclosure relates to methods of treating acute myeloid leukemia (AML)comprising administering to a patient in need thereof an effective amount of iron, heme, or other iron complexing agent and obatoclax in an amount of 5 mg daily for four or more weeks.

In certain embodiments, this disclosure relates to methods of treating acute myeloid leukemia (AML) comprising administering to a patient in need thereof an effective amount of iron, heme, or other iron complexing agent and obatoclax in an amount of 10 mg daily for two or more weeks. In certain embodiments, this disclosure relates to methods of treating acute myeloid leukemia (AML) comprising administering to a patient in need thereof an effective amount of iron, heme, or other iron complexing agent and obatoclax in an amount of 10 mg daily for three or more weeks. In certain embodiments, this disclosure relates to methods of treating acute myeloid leukemia (AML)comprising administering to a patient in need thereof an effective amount of iron, heme, or other iron complexing agent and obatoclax in an amount of 10 mg daily for four or more weeks.

In certain embodiments, this disclosure relates to methods of treating acute myeloid leukemia (AML) comprising administering to a patient in need thereof an effective amount of iron, heme, or other iron complexing agent and obatoclax in an amount of 20 mg daily for two or more weeks. In certain embodiments, this disclosure relates to methods of treating acute myeloid leukemia (AML) comprising administering to a patient in need thereof an effective amount of iron, heme, or other iron complexing agent and obatoclax in an amount of 20 mg daily for three or more weeks. In certain embodiments, this disclosure relates to methods of treating acute myeloid leukemia (AML) comprising administering to a patient in need thereof an effective amount of iron, heme, or other iron complexing agent and obatoclax in an amount of 20 mg daily for four or more weeks.

In certain embodiments, this disclosure relates to methods of treating acute myeloid leukemia (AML) comprising administering to a patient in need thereof an effective amount of iron, heme, or other iron complexing agent and obatoclax in an amount of 40 mg daily for two or more weeks. In certain embodiments, this disclosure relates to methods of treating acute myeloid leukemia (AML) comprising administering to a patient in need thereof an effective amount of iron, heme, or other iron complexing agent and obatoclax in an amount of 40 mg daily for three or more weeks. In certain embodiments, this disclosure relates to methods of treating acute myeloid leukemia (AML) comprising administering to a patient in need thereof an effective amount of iron, heme, or other iron complexing agent and obatoclax in an amount of 40 mg daily for four or more weeks.

Iron Supplementation Selectively Induces Ferroptosis in Venetoclax Sensitive t(11; 14) Multiple Myeloma

Low electron transport chain (ETC) activity and reduced oxidative phosphorylation (OXPHOS) in a patient with multiple myeloma are indicative that the patient will respond to venetoclax therapy. Experiments were performed to determine whether iron supplementation and chelation effect the growth of multiple myeloma cells and sensitivity to venetoclax therapy. Iron supplementation was found to have growth inhibitory effects in venetoclax sensitive t(11; 14) multiple myeloma cell lines while promoting proliferation of non t(11; 14) multiple myeloma cell lines. Iron supplementation paired with low doses of venetoclax exhibited overall greater cytotoxicity than the individual agents in t(11; 14) multiple myeloma cell lines.

Experiments also indicate that chelating iron uniformly sensitized both t(11; 14) and non-t(11; 14) multiple myeloma cells. Experiments using iron supplementation to induce venetoclax sensitization indicated iron suppressed expression of the antioxidant protein GPX4 and induced mitochondrial ROS, suggestive of ferroptosis. Blocking ferroptosis by a synthetic antioxidant, ferrostatin was sufficient to mitigate the iron mediated toxicity in t(11; 14) multiple myeloma cell lines. Although it is not intended that embodiments of this disclosure be limited by any mechanism, it is contemplated that t(11; 14) multiple myeloma cells are unable to handle excess iron leading to elevated cytotoxicity when combined with venetoclax suggesting iron supplementation as a translationally implementable strategy to increase venetoclax sensitivity in patients with t(11; 14) multiple myeloma or other cancers.

Venetoclax is a BH3 mimetic that inhibits the anti-apoptotic protein, B cell lymphoma-2 (BCL-2). In many patients with a t(11; 14) translocation, venetoclax is effective as a single agent. Treating multiple myeloma patients using venetoclax in combination strategies often results in relapse to refractory disease within approximately a year of treatment. Normal peripheral B cells are also sensitive to venetoclax underscoring the need for reducing venetoclax dosing-associated toxicities and development of biomarker-driven therapeutic strategies for identification of patients that benefit from anticancer therapies.

Experiments reported herein indicate that reduced ETC activity in venetoclax sensitive multiple myeloma renders these cells sensitive to iron supplementation and iron supplementation promotes GPX4-dependent ferroptosis. Combination of iron with venetoclax in the single agent for treatment of venetoclax sensitive cells allows for administration of venetoclax at lower IC₅₀ doses providing a strategy for reducing dose-associated toxicities in t(11; 14) MM in addition to overcoming resistance-promoting antioxidant effects.

In multiple myeloma, venetoclax is effective as a single agent in a subset of patients exhibiting the t(11; 14) translocation and has efficacy as salvage therapy in combination with other drugs. Glucocorticoids and PIs such as bortezomib and dexamethasone and carfilzomib and dexamethasone are contemplated to be administered in combination with venetoclax and other BCL-2 inhibitors. Experiments indicated progression free and overall survival in t(11; 14) patients with high BCL-2 expression. Increased mortality in the non-t(11; 14) MM may be attributed to increased incidence of infection and myelosuppression.

Venetoclax in combination with other agents may have adverse effects related to pancytopenia, electrolyte imbalance tumor lysis syndrome, and respiratory infections. Thus, there is a need to identify strategies to reduce side effects and to devise combination strategies that that elicit desirable responses.

### T(11; 14) MM Are Sensitive to Iron Supplementation

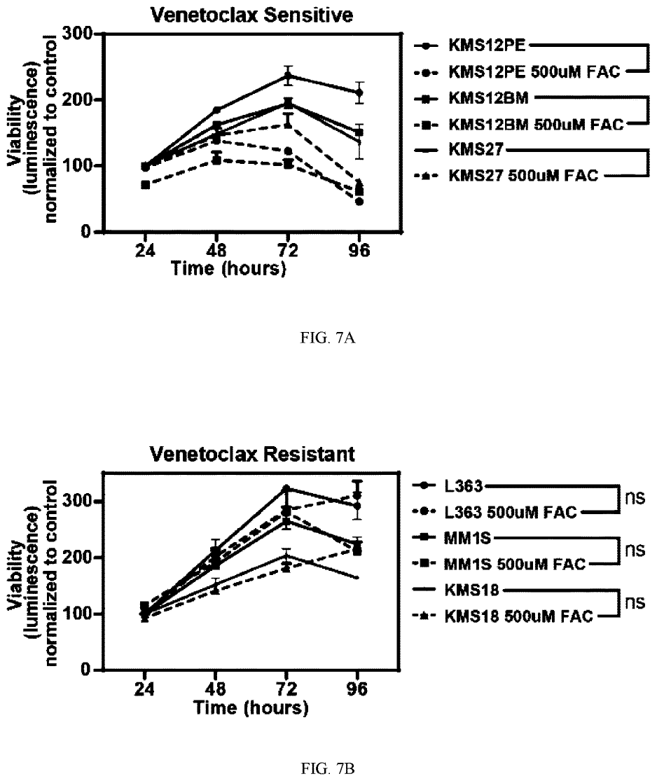

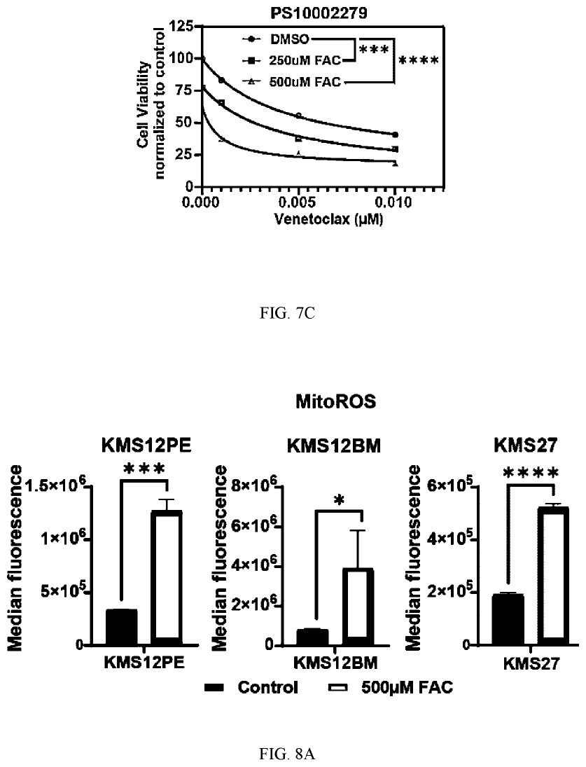

Venetoclax sensitive (VS) MM cells exhibit reduced electron transport chain (ETC) complex I and II activity and oxidative phosphorylation (OXPHOS). Iron is a cofactor for the maintenance of ETC activity. The impact of modulating iron levels either by chelation with deferoxamine (DFO) or supplementation with ferric ammonium citrate (FAC) was assessed in a panel of VS t(11; 14) and VR non t(11; 14) MM cell lines. Interestingly, while iron chelation had growth inhibitory effects across both VS and VR MM cell lines, iron supplementation was found to specifically inhibit the proliferation of VS cell lines, as shown in FIG. 7A.

### Iron Supplementation Enhances Sensitivity of T(11; 14) MM to Venetoclax

Since there was a growth suppressive effect of FAC in t(11; 14) MM that are normally sensitive to single agent venetoclax, the effects of administering venetoclax with iron supplementation was tested. Enhanced sensitivity to venetoclax was detect in a dose-dependent manner in all the VS t(11; 14) cell lines tested, even at 10 nM venetoclax, which is a significantly lower dose than the reported IC₅₀ of venetoclax about 100 nM. A similar response was observed in a t(11; 14) patient sample (FIG. 7C). Iron supplementation, however, had no impact on the response of the VR non t(11; 14) cell lines to venetoclax. The protein expression levels of BCL-2 family members in VS t(11; 14) MM cells were treated with and without FAC.

FAC Treatment Induces Mitochondrial-ROS and Lipid Peroxidation in t(11; 14) VS MM

Free or excess intracellular iron is a potent inducer of reactive oxygen species (ROS) and oxidative stress. Evaluation of mitochondrial ROS in FAC treated VS t(11; 14) MM, demonstrated significant elevation in mitochondrial ROS and not in cytosolic ROS levels (FIG. 8A). Supportive of this, a consistent reduction of glutathione peroxidase 4 (GPX4)—a glutathione (GSH)-dependent antioxidant enzyme, was observe across all VS t(11; 14) cell lines upon FAC treatment. The cystine-glutamate antiporter SLC7A11 was not altered with FAC treatment. In sum, these results suggest FAC induces ferroptosis in venetoclax sensitive t(11; 14) MM cells. Evaluation of protein expression of other key players linked to iron metabolism, including the iron importers transferrin (TFR1), and DMT1, exporter (ferroportin), and storage proteins such as ferritin(FTH), indicate an increase in iron storage protein ferritin leading to a reduction of the iron importer TFR1 in response to the excess iron accumulation due to FAC treatment. Overall, these results suggests that a dysfunctional xCT/GPX4 axis in VS t(11; 14) MM cells render t(11; 14) VS MM incapable of handling the excessive iron induced-mitochondrial ROS generated upon FAC treatment.

Inhibiting Ferroptosis Rescues FAC Mediated Cell Death in VS t(11; 14)

Intracellular iron overload, ROS mediated lipid peroxidation, and loss of GSH/GPX4 mediated antioxidant defenses are all features of ferroptosis. Experiments were performed to determine whether FAC supplementation in VS t(11; 14) MM promotes cell death via ferroptosis. VS t(11; 14) cell lines were treated with a ferrostatin (Fn-1), a synthetic antioxidant that acts via a reductive mechanism to prevent damage to membrane lipids and thereby, inhibits cell death. Inhibiting ferroptosis was able to fully rescue the cell death induced by FAC both in the presence and absence of venetoclax (FIG. 8B). Boc-D-FMK is a caspase inhibitor that inhibits apoptosis. Boc-D-FMK, however, was not able to rescue FAC mediated cell death. These results indicate that FAC supplementation in VS t(11; 14) MM induces ferroptosis.

### Reduced ETC Activity Correlates With Venetoclax Sensitivity

Experiments indicate that venetoclax sensitivity and cellular energetics are coupled to basal, spare and maximal respiration and electron transport chain (ETC) complex I and II activity and are significantly reduced in venetoclax sensitive multiple myeloma (VS MM), irrespective of the t(11; 14) status. Heme is involved maintaining ETC activity and consequently OXPHOS, prompting experiments into the role of dysregulated heme homeostasis in MM Ven sensitivity. VS MM exhibit reduced heme biosynthesis, elevated heme importer and exporter gene expression. MM can be characterized into 7 disease subtypes based on gene expression, correlating with the occurrence of hyperdiploidy, and recurrent translocations. MM t(11; 14) has two gene expression subgroups, CD1 and CD2, that both exhibit translocation between CCND1 and the immunoglobulin heavy chain locus. The CD2 subgroup is the MM subset where venetoclax is most effective, thus allowing for query of the CD2 subtype as a proxy for venetoclax sensitivity in MM. CD2 vs CD1 MM exhibit reduced ETC/OXPHOS gene signatures. Expression of heme biosynthetic genes in CD2 vs CD1 MM were examined using a clinical trial dataset (NCT0145429). Heme biosynthesis gene expression of ALAS1 and FECH were significantly suppressed. Evaluation of protoporphyrin X (PpIX) the penultimate intermediate of heme synthesis indicates VS MM exhibits significantly reduced PpIX. Supplementation with 5-ALA, a heme biosynthetic precursor that is metabolized to fluorescent PpIX97, shows significantly lower fluorescence in VS t(11; 14) relative to VR MM lines. Supplementation with heme shows VS MM to exhibit significantly higher heme uptake compared to VR cells in line with reduced heme biosynthesis, indicative of a lower PpIX biosynthetic capacity and that is consistent with the diminished expression of heme biosynthetic enzymes from the CD2 VS group. Evaluation of heme gene expression indicates heme-related gene signatures are elevated in relapse refractory (RR) MM, underscoring potential relevance of increased heme levels in MM relapse.

Hemin Supplementation Reverses Ven Sensitivity in t(11; 14) MM

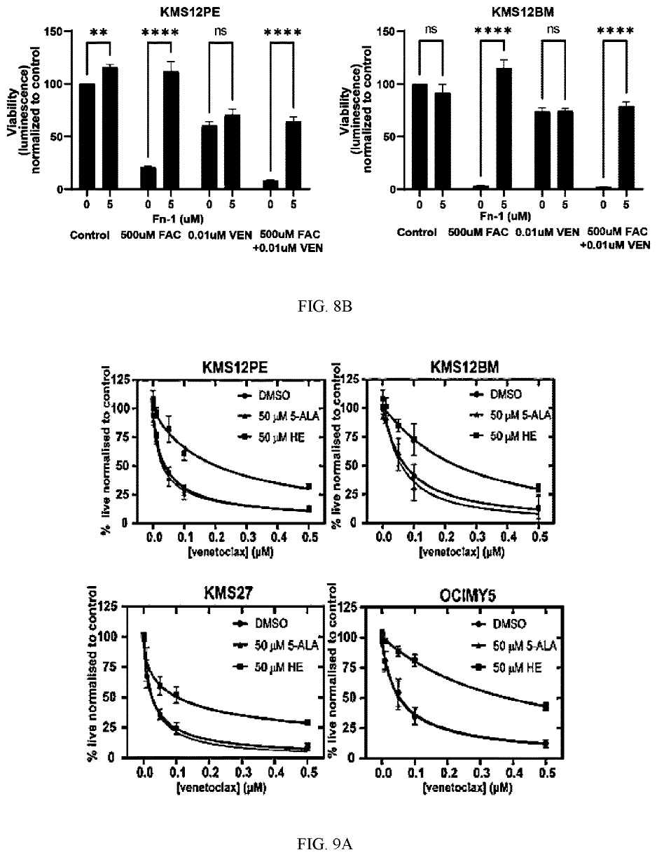

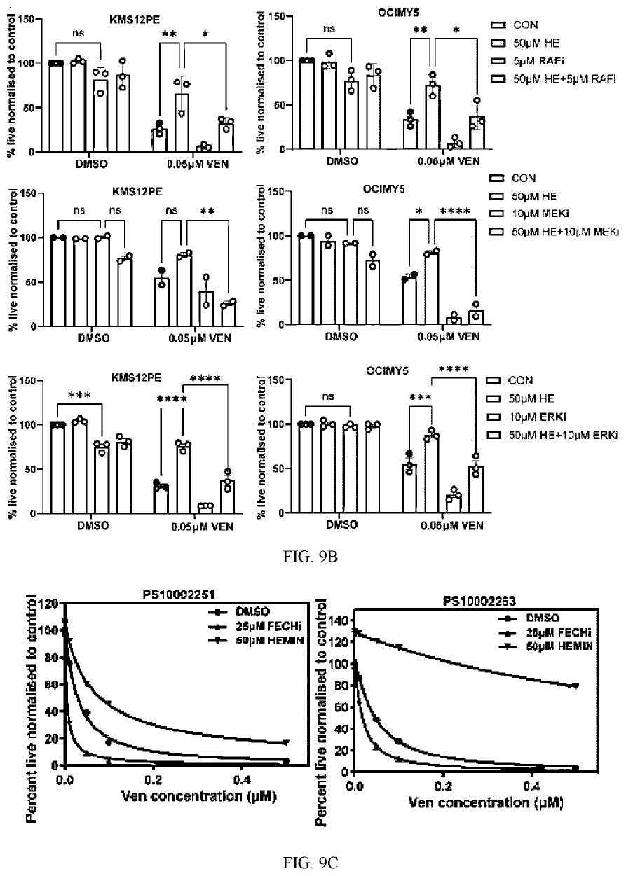

To test the necessity for reduced heme synthesis for Ven sensitivity in t(11; 14) MM, Ven treated cells were supplemented with either the heme synthesis precursor 5-ALA or hemin (oxidized heme and active ingredient of clinically approved Panhematin™). 5-ALA supplementation did not rescue Ven sensitivity; however, hemin supplementation significantly reversed Ven sensitivity in all VS MM (FIG. 9A).

Experiments were performed to determine whether activation of kinases is involved in hemin-induced resistance to Ven. Co-treatment with specific inhibitors of RAF, MEK and ERK reversed hemin-induced resistance (FIG. 9B).

In certain embodiments, this disclosure contemplates that t(11; 14) venetoclax sensitive MM exhibit reduced heme synthesis and elevated sensitivity to iron supplementation. Thus, methods of treating MM or other hematological malignancies can be improved by including exogenous iron, heme, iron complex or salt thereof.

In certain embodiments, this disclosure contemplates that hemin supplementation promotes resistance to venetoclax that can be reversed upon inhibition of the RAS-RAF-MEK-ERK pathway with various clinically approved inhibitors.

In certain embodiments, this disclosure contemplates that hemin can be used in combination with venetoclax to increase venetoclax sensitivity.

In certain embodiments, this disclosure contemplates that reduced heme levels in t(11; 14) MM makes these MM cells vulnerable to ferroptosis with supplemented iron or to SLC7A11 inhibition with erastin; while ETC suppressed non t(11; 14) are sensitized to erastin upon ETC inhibition leading to increased ferroptosis.

In certain embodiments, this disclosure contemplates that ferrochelatase inhibition sensitizes MM to venetoclax.

In certain embodiments, this disclosure contemplates that chelation sensitizes non t(11; 14) MM to venetoclax.

In certain embodiments, this disclosure contemplates that ability of protoporphyrin levels to distinguish VS verses venetoclax resistant (VR) MM allows for detection of VR MM by measuring protoporphyrin levels.

In certain embodiments, this disclosure contemplates that inability of VS t(11; 14) MM to handle excess iron indicates the use of iron supplementation to increase venetoclax sensitivity in the broader MM population.

In certain embodiments, this disclosure contemplates that administering a second agent that further sensitizes t(11; 14) MM to venetoclax allows for administering venetoclax at lower doses for a longer duration to elicit deeper durable responses with reduced venetoclax toxicities as normal peripheral B cells are sensitive to venetoclax providing dose-reducing associated toxicities. In certain embodiments, this disclosure contemplates the use of iron supplementation agents such as ferrous sulfate and ferumoxytol.

In certain embodiments, this disclosure contemplates that iron supplementation prevents acquired resistance to venetoclax and induce ferroptosis.

In certain embodiments, this disclosure contemplates that MM tumors are heterogenous in their expression/dependence on BCL-2 and ETC gene expression supporting the use combination therapies to increase BCL-2 dependency.

1-8. (canceled)

9. A method of treating cancer comprising administering to a patient in need thereof an effective amount of a BCL-2 inhibitor in combination with heme or hemin.

10. The method of claim 9, wherein administering to a patient in need thereof an effective amount of a BCL-2 inhibitor in combination with heme or hemin is in combination with iron, iron complexing agent or salt thereof.

11. The method of claim 9, wherein the BCL-2 inhibitor is venetoclax.

12. The method of claim 9, wherein the cancer is multiple myeloma.

13. method of claim 9, wherein the patient is diagnosed with a t(11; 14) translocation.

14. The method of claim 9, wherein the BCL-2 inhibitor is administered in combination with a MEK inhibitor.

15. The method of claim 14, wherein the administration of the BCL-2 inhibitor and the MEK inhibitor are in combination with heme or hemin.

16. The method of claim 9, wherein the BCL-2 inhibitor is administered at a lower amount when compared to the amount normally clinically administered considering the weight of the patient.

17. A method of treating cancer comprising administering to a patient in need thereof an effective amount of a BCL-2 inhibitor in combination with a SLC7A11 inhibitor.

18. The method of claim 17, wherein the SLC7A11 inhibitor is erastin.

19. The method of claim 17, wherein the BCL-2 inhibitor is venetoclax.

20. The method of claim 17 wherein administering to a patient in need thereof an effective amount of a BCL-2 inhibitor and a SLC7A11 inhibitor is in combination with administering heme or hemin and iron or iron salt.

21. A method of diagnosing and treating a patient with cancer comprising diagnosing whether the patient has low levels of aminolevulinic acid (ALA) by obtaining a sample from the patient and detecting in the sample low levels of aminolevulinic acid (ALA) providing a diagnosis of low levels of aminolevulinic acid (ALA); and
if the subject is diagnosed with low levels of aminolevulinic acid (ALA), administering to the patient an effective amount of a BCL-2 inhibitor in combination with heme, hemin, iron, iron complex, or iron salt.

22. The method of claim 21, wherein the BCL-2 inhibitor is venetoclax.
